(19) United States
(12) Patent Application Publication
(10) Pub. No.: US 2023/0009794 A1
(43) Pub. Date: Jan. 12, 2023

(54) ACCOMMODATIVE INTRAOCULAR IMPLANT WITH SELF-ADJUSTABLE SIZING

(71) Applicant: Valdemar Portney, Newport Coast, CA (US)

(72) Inventor: Valdemar Portney, Newport Coast, CA (US)

(21) Appl. No.: 17/810,633

(22) Filed: Jul. 4, 2022

Related U.S. Application Data
(60) Provisional application No. 63/203,124, filed on Jul. 9, 2021.
(60) Provisional application No. 63/203,443, filed on Jul. 22, 2021.

(51) Int. Cl. A61F 2/16; A61F 2/48

(52) U.S. Cl. CPC A61F 2/1635; A61F 2/482; A61F 2250/0003; A61F 2002/1689; A61F 2002/1682; A61F 2250/0097

(57) ABSTRACT

An accommodative intraocular lens includes an optic body having an optical power changing structure. There are two supporting structures disposed opposite one another about the optic body, each supporting structure configured to be connected at a distal end to a ciliary body after implantation in an eye of a patient and connected at a proximal end to the optic body. The two supporting structures are a zeroing supporting structure and an actuating supporting structure. The zeroing supporting structure is configured to not change the optical power of the optic body in an installation mode and an operation mode of the accommodative intraocular lens. An actuating supporting structure is configured to not change the optical power of the optic body in the installation mode but is configured to change the optical power of the optic body in the operation mode of the accommodative intraocular lens.

### CROSS-REFERENCE TO RELATED APPLICATIONS

This application claims priority to provisional application No. 63/203,124 filed on Jul. 9, 2021 and provisional application No. 63/203,443 filed on Jul. 22, 2021, the entire contents of which are fully incorporated herein with these references.

### DESCRIPTION

### Field of the Invention

The present invention relates generally to an accommodative intraocular lens (AIOL) placed inside the eye to create an image at the retina of objects at different distances from the eye by changing lens power under the action of eye's ciliary muscle.

### Background of the Invention

An accommodative intraocular lens (AIOL) relies on ciliary muscle topographic change for its optical power change. The AIOL can be a fluidic lens and its power changes by a change of the lens shape, by moving high power lens along the optical axis in dual-lens construction or rely on Alvarez principle where two wave plates perpendicular to optical axis are mutually shifted to change a focus position. The representative prior art describing different types of accommodative IOL are the following and are incorporated herein by these references: U.S. Pat. No. 11,109,960 by Borja, et al.; U.S. Pat. No. 10,285,805 by de Juan, Jr., et al.; 11,065,107 by Brady, et al.; U.S. Pat. No. 10,052,194 by Bumbalough; and U.S. Pat. No. 10,463,473 by Rombach and Simonov.

An AIOL can be a switchable lens where the power changes by switching between refractive and diffractive optical states through a diffractive index control by electric field or with a fluid transfer. For instance, a paper by a group of researchers taught an application to the spectacle lens but it can also be applied to an intraocular lens. See Li G., Mathine D. L., Valley P., et al., “Switchable electro-optic diffractive lens with high efficiency for ophthalmic application”, Proceedings of the National Academy of Science of the USA, 2006; 103: 6100-6104, the contents of this paper are herein incorporated in full with this reference. The operation of the described by Li G, et al. spectacle lens was based on electrical control of the refractive index of thin layer of pneumatic liquid crystal by creating volume diffractive element through refractive index modulation. Another type of switchable optic was described by Portney in U.S. Pat. No. 9,364,319, the contents which are herein incorporated in full with this reference. It described refractive-diffractive switchable lens that creates an image at a position produced by the lens in diffractive state that is different from the image position produced by the lens in refractive state by changing between refractive and diffraction surface elastic membrane shape over the optical substrate with minute amount of fluid transfer within the switchable lens.

All referenced above AIOL are called opto-mechanical lenses and ciliary muscle acts as ocular actuator to power the AIOL optic for an optical power change between the eye's dis-accommodation and accommodation states.

AIOL can be an opto-electronic type where its optical power changes under the action of an engineering actuator installed at the implant triggers by ciliary muscle change with eye accommodation. As an example, but not limited to it, an engineering actuator may be a system for switching electrical field to change a refractive index of an optical part of the implant as described above or fluid transfer in opto-fluidic lens per above description. Such lenses are called opto-electronic lenses. In such lenses a topographic change of ciliary muscle is sensed and prompts the engineering actuator to change an electric field for the refractive index change or a fluid transfer for a lens power change.

An opto-mechanical lens described above might be converted into an opto-electronic lens by installing an engineering actuator (piezo-electric, electro-magnetic, electro-static, ionic polymer-metal composites, so on) at the implant to power the optical part for the optical power change. In this case the ciliary muscle topographic change (accommodation sensing) is sensed by a pressure detector to communicate with the engineering actuator to actuate the optical power change.

One of the challenges in utilizing topographic changes of the ciliary body for actuation of an opto-mechanical lens or sensing in an opto-electronic lens, is an individual variation of ciliary body ring diameter (CBR_{D}) at dis-accommodation when the ciliary muscle is relaxed, which is the so called static ciliary body ring diameter (CBRD_{S}) variation between the patients. The challenge is to match implant overall size (diameter) and ciliary body ring diameter of an individual eye at dis-accommodation state upon the implant installation in the eye in order to reliably respond to a ciliary body ring diameter change with eye's accommodation. A response to accommodation involves ciliary body ring diameter reduction with ciliary muscle contraction, so called dynamic ciliary body ring diameter (CBRD_{D}), in order for implant optical power change in coordination with a difference (CBRD_{S}−CBRD_{D}). The challenge is that the individual variation of static ciliary body ring diameter CBRD_{S} is similar in magnitude to a difference (CBRD_{S}−CBRD_{D}) occurred with eye accommodation thus making a distinction between a power change of implant's optic body due to either individual CBRD_{S} variation or CBR_{D} change with accommodation difficult if not impossible.

Adnan Khan's data (Ref. 1) references to CBRD_{S}=11.07±0.47 (mm) without accommodation (dis-accommodation) and CBRD_{D}=10.64±0.44 (mm) with accommodation, Richdale's data (Ref. 2) references to CBRD_{S}=11.62±0.37 (mm) without accommodation (dis-accommodation) and CBRD_{D}=11.47±0.37 (mm) with 4 D accommodation. Thus, static ciliary body ring diameter (CBRD_{S}) variation between individuals is similar to a magnitude of reduction in ciliary body ring diameter with accommodation, i.e., a difference (CBRD_{S}−CBRD_{D}). Additional factors contribute to matching the implant size to CBRD_{S} is a variation in lens position such as implant tilt or centration inside the eye. This creates additional discrepancy between AIOL sizing required to match CBRD_{S} dimension in order to responded to a CBR_{D} change with accommodation.
Reference 1: Khan A, Pope J M, Verkicharla P K, Suheimat M and Atchison D A. Change in human lens dimensions, lens refractive index distribution and ciliary body ring diameter with accommodation. Biomed Opt Express 2018; 9(3):1272-1282.
Reference 2: Kathryn Richdale, The Human Eye in Presbyopia: Changes in the Lens and Ciliary Body with Age and Accommodation., DISSERTATION Presented in Partial Fulfillment of the Requirements for the Degree Doctor of Philosophy in the Graduate School of The Ohio State University, The Ohio State University 2011. Reference 1 and 2 are fully incorporated herein with these references.

Such individual variation in CBRD_{S} and mismatch with the implant sizing may cause different patient-to-patient “static pressure” on the implant which might interpreted as a pressure by the ciliary body on the implant in dis-accommodation state. A variation in static pressure may create a power change of the AIOL without an accommodation response potentially effecting individual patient vision at dis-accommodation, i.e., at far focus, due to inducing refractive error.

Another outcome of the mismatch between implant sizing and CBRD_{S} effects a reliability of the implant optical power response to a change in accommodation state. If the static ciliary body ring diameter is unpredictable for a static pressure, a difference (CBRD_{S}−CBRD_{D}) leads to an unpredictable range of pressure change on the implant, and, therefore, optical power response is also unpredictable. An AIOL design intend is to manifest a correlation between the ocular actuation in terms of a difference (CBRD_{S}−CBRD_{D}) with accommodation and an optical power elevation to create a predictable increase in optical power. If the CBRD_{S} is unpredictable as a pressure on the implant at dis-accommodation is uncertain, and therefore, a change in pressure on the implant due to a difference (CBRD_{S}−CBRD_{D}) with accommodation is also unpredictable resulting in uncertainty of the optical power change with accommodation.

In case of a lens with power switching, the performance is expected to be more reliable because of a binary nature of the correlation between pressure on implant due to a difference (CBRD_{S}−CBRD_{D}) and optical power change; if a pressure difference is above a set level, the switching occurs to the elevated optical power of the accommodation state. If the pressure difference is below a set level, the optical power switches back to a preset level for the dis-accommodation state.

In order to manifest a predictable static pressure regardless of the individual ciliary body ring diameter at dis-accommodation, so called “size zeroing” must be a part of AIOL installation inside the eye. Size zeroing is defined as matching the overall size of the AIOL to the CBRD_{S} to manifest a predictable by the implant design static pressure on the AIOL in the dis-accommodation state. Static pressure cannot be zero because the AIOL is supported by the ciliary body inside the eye, but it shall maintain a certain predictable by the implant mechanical design level of pressure on the AIOL that secures the implant position inside the eye. As a result, the static pressure at dis-accommodation is predictable and, therefore, an individual pressure change due to a difference (CBRD_{S}−CBRD_{D}) with accommodation is also becomes predictable to allow a coordination between AIOL optical power change and accommodation response upon ciliary muscle contraction.

Therefore, a need exists for sizing self-adjustment mechanism for AIOL for matching with an individual eye ciliary body ring diameter at dis-accommodation for the affective operation of an AIOL with eye accommodation.

### SUMMARY OF THE INVENTION

An AIOL of the current invention is placed inside the eye for power change by interacting with ciliary body with eye accommodation. The AIOL consists of optic body that includes power changing structure for changing optical power of the optic body. Power changing structure may operate by different means: fluidic shape changing, per Alvarez principle, double lens relative movement, opto-electric operation such as material refractive index change with electric field, opto-fluidic switching and so on. The AIOL includes three interactive structures: (1) a supporting structure to support the optic body inside the eye by the ciliary body directly or indirectly via a lens bag; (2) a modifiable structure to communicate between the supporting structure and power changing structure, where the modifiable structure includes a locking structure for modification of the modifiable structure between changeable and unchangeable states; and (3) a power changing structure to change a power of the optic body.

The supporting structure includes supporting element which is in contact with eye's ciliary body and deformable element which deforms due to ciliary body pressure change on the supporting element with a change in ciliary body ring diameter (CBR_{D}) resulting in changing shape of the supporting structure. There are different types of deformable elements. For instance, a deformable element in a form of elastic fiber loop that deforms to change its shape with CBR_{D} change, or a fluidic chamber that deforms with changing its volume as CBR_{D} changes—they are in so called mechanical AIOL where interaction between the supporting and modifiable structures are mechanical in nature. A deformable element might be a pressure sensor that deforms to generate an electrical signal with CBR_{D} change—it is in so called electronic AIOL where interaction between the supporting and modifiable structures are electronic in nature. A supporting structure may also include a combination of different type of deformable elements, for instance, elastic fiber loop and fluidic change, elastic fiber look and pressure sensor, and so on.

The modifiable structure communicates between the supporting structure and power changing structure by changing one of its characteristics such as position, shape, fluid flow and electrical signal depending upon a type of deformable element of the supporting structure. For instance, if a deformable element is an elastic fiber which deforms with supporting structure shape change under CBR_{D} change, it in turn changes a position in or within the modifiable structure which may be in a form of a rounded or rectangular cylinder; or if a deformable element is a fluid chamber which deforms with corresponding supporting structure shape change with CBR_{D} change, it in turn creates a fluid flow in or within the modifiable structure which might be in a form of a hollow tube or membrane; or if a deformable structure includes a pressure sensor which deforms with CBR_{D} change, it in turn varies an electric signal in or within of modifiable structure as being an electronic device. Correspondently, cylinder position change impact a power structure to change optic body power with CBR_{D} change, or fluid flow impacts a power structure to change optic body power with CBR_{D} change, or electric signal variation impacts a power structure to change optic body power with CBR_{D} change. A modifiable structure may also be a combination of above characteristics.

A change of AIOL sizing with CBR_{D} occurs due to two factors—a variability between different patients or due to accommodation response at the individual patient. In order to coordinate an AIOL size adjustment initially to match the CBR_{D} of an individual patient without interaction with a power structure of the optic body and then the AIOL size adjustment with interaction with a power structure for optic body power change with CBR_{D} change due to accommodation, the modifiable structure as well as the corresponding AIOL undertake three consecutive states. (1) First, a changeable state in which it changes in one of a position, shape, fluid flow and electric signal in or within it with a size change of AIOL as supporting structure changes with CBR_{D} change and without interaction with a power structure of optic body—it is called first changeable state, it is to manage CBR_{D} variability between the patients. (2) Second, an unchangeable state if none of the characteristics such as position, shape, fluid flow and electric signal in or within of the modifiable structure changes and the modifiable structure forces the connected supporting structure to maintain its shape and form. It is a transitional step to stabilize the AIOL in conventional form of IOL where no change in any AIOL structure occurs. (3) Third, a changeable state in which it changes one of a position, shape, fluid flow and electric signal in or within it with a shape change of the supporting structure of AIOL as supporting structure changes with CBR_{D} and interacts with a power changing structure of optic body to change optic body power. It is to allow for AIOL accommodation—it is the second changeable state. Thus, the AIOL undertakes two modes: an installation mode where an ophthalmologist is involved. It starts with modifiable structure and thus the AIOL being in first changeable state and ends up with the second modification into the second changeable state while an ophthalmologist installs the AIOL in the eye and instigates first and second modifications of the modifiable structure and this AIOL; and an operation mode starting with the AIOL becoming into the second changeable state in which the patient utilizes the AIOL for patient's visual needs.

First changeable state results in matching a size of the AIOL to the individual CBR_{D} at dis-accommodation by sizing self-adjustment. During this process the modifiable structure of a free standing AIOL in first changeable state where no interaction with power changing structure occurs and the patient being in dis-accommodating state, for instance by pharmaceutically relaxing the ciliary muscle. In order for CBR_{D} to effect supporting structure shape, a diameter of a free standing AIOL of the present invention is larger than a high end of CBRD_{S} of the population, say 12-13 mm. Free standing AIOL means a lens being outside the eye before its implantation.

A locking structure of the AIOL services for modification of the modifiable structure between changeable and unchangeable states. Thus, a modifiable structure is characterized by a changeable characteristic such as a change of one of a position, shape, fluid flow and electric signal in or within it, and the locking structure is applied either to terminate the ability of the modifiable element to change the characteristic or initiate the ability to change the characteristic. A locking structure is controlled by external means by an ophthalmologist or other provider, either by a mechanical probe or laser beam if a mechanical modification of the modifiable structure is involved or by a wireless radio signal in case of electronic communication. The first modification of the modifiable structure of the AIOL is from first changeable state into unchangeable state where the AIOL size is fixed at matching individual CBRD_{S} at dis-accommodation without impacting a power changing structure of the AIOL. It is followed by second modification from unchangeable state to the second changeable state where size variation of the AIOL impacts power changing structure for power change. As a result of the described above installation mode, self-adjustable AIOL of the present invention ends up in size matching to the individual patient CBRD_{S} in dis-accommodation state and coordinating power change of the AIOL optic body with CBRD_{D} change due to eye accommodation.

Modifiable structure may compose of a single unit or two-unit composition which depends on a modification characteristic between the changeable and unchangeable states, i.e., one of a position, shape, fluid flow and electric signal. Two-unit composition is involved if a mechanical modification such as one of position, shape and fluid flow is required in or within the modification structure. Singe-unit composition might be adequate for electronic modification where modification structure is characterized by electric signal change because electric signal is highly controllable with programming the modification structure by an ophthalmologist via the locking structure—a different wireless radio signals utilized for communicating with locking structure allows to distinguish first and second modifications between first changeable state, unchangeable state and second changeable state in the electronic system of the modifiable structure.

Each unit of two-unit composition of the modifiable structure services either first or second modification. The unit involved in first modification is called zeroing modifiable structure and all structures and elements connected to it are also called zeroing, i.e., zeroing supporting structure, zeroing stopper, and so on. The unit involved in second modification is called actuating modifiable structure and all structures and elements connected to it are also called actuating, i.e., actuating supporting structure, actuating stopper, and so on.

The application of self-adjusting configuration is applied to opto-fluidic switchable AIOL which incorporates switchable optical element (SOE) described by the US Patent Application No: 2021/0240010 by Portney. Below is a description of SOE. A combination of elastic membrane and optical substrate is attached to the substrate support and membrane cover via elastic elements at their peripheries to allow optical substrate-membrane combination to displace inside the switchable optical element. A non-matching fluid occupies the external chamber between membrane cover and the membrane. Matching fluid occupies active chamber between membrane and substrate and internal chamber between substrate and substrate cover. Active and internal chambers are connected by multiple holes through the substrate. In the refractive state the elastic membrane takes a refractive continuous surface shape characterized by a surface curvature with matching fluid in the active chamber masking the diffractive surface of the optical substrate. A diffractive surface is characterized by periodic diffractive grooves, usually a blazed shape. In this refractive state the SEO manifest a certain power, usually for far focus. As the optical substrate and membrane combination can be displaced towards the membrane cover due to a support by elastic elements within the SOE, an elevation of pressure in the external chamber forces the elastic membrane take a shape of the substrate diffractive surface by squeezing out a matching fluid from the active chamber. As a result, the SOE takes a power of the diffractive surface defined by the periodicity of the diffractive grooves which usually higher to provide intermediate or near focus.

In order to conduct substrate-membrane displacement, the substrate is extended with a substrate extension outside the SOE chambers, i.e., outside the elastic elements supporting the substrate-membrane combination. The opto-fluidic switchable AIOL includes two-unit modifiable structure because it relies on mechanical actions for first and second modifications—one unit is zeroing structure, and another is actuating structure. The zeroing structures and elements are engaged in first modification of the AIOL for size matching with individual patient CBRD_{S} at dis-accommodation state. The actuating structures and elements are engaged in second modification for power switching by SOE with CBRD_{D} change with accommodation. The exposure of the substrate extension outside the SOE chambers of the AIOL allows for its interaction with an actuating modifiable structure in the second changeable state to coordinate SOE power switching with eye accommodation, i.e., switching between refractive and diffractive states of different powers with a change in CBRD_{D} with eye accommodation and dis-accommodation.

### BRIEF DESCRIPTION OF THE DRAWINGS

The advantages and features of the present invention will be better understood by the following description when considered in conjunction with the accompanying drawings in which:

FIG. 1 shows a front view of an AIOL in free standing state. it includes optic body with a power changing structure and supporting structures with deformable fiber loops in the composition of two units—zeroing structure for sizing self-adjustment and actuating structure for accommodation response.

FIG. 2 demonstrate an example of zeroing modifiable structure in a form of zeroing pusher at the AIOL per FIG. 1.

FIG. 3 demonstrates an example of actuating modifiable structure in a form of actuating pusher at the AIOL per FIG. 1.

FIG. 4 shows sizing self-adjustable AIOL of FIG. 1 in installation mode where the AIOL is placed inside the eye and its size is self-adjusted to an individual ciliary body ring diameter at dis-accommodation state, CBRD_{S}. The modifiable structure is in first changeable state where only the zeroing modifiable structure is acting for AIOL sizing self-adjustment without interacting with power changing structure.

FIG. 5 shows the AIOL of the FIG. 4 in Installation mode where the modifiable structure has undergone first modification and the corresponding AIOL comes into unchangeable state.

FIG. 6 shows the AIOL of the FIG. 5 in operation mode where the modifiable structure has undergone second modification to come into second changeable state where only actuating modifiable structure changes with accommodation. It also interacts with power changing structure to change optic body power with eye accommodation and dis-accommodation.

FIG. 7 shows sizing self-adjustable AIOL where the supporting structure includes deformable element as variable fluidic chamber. The AIOL is in the installation mode where the AIOL is placed inside the eye and its size is self-adjusted to an individual ciliary body ring diameter at dis-accommodation state, CBRD_{S}. The modifiable structure and the corresponding AIOL are in first changeable state where only the zeroing modifiable structure is acting for AIOL size self-adjustment without interacting with the power changing structure of the optic body.

FIG. 8 shows the AIOL of the FIG. 7 in installation mode where the modifiable structure has undergone first modification and corresponding AIOL to come into unchangeable state.

FIG. 9 shows the AIOL of the FIG. 5 in operation mode where the modifiable structure and the correspond AIOL are in second changeable state upon second modification where only actuating modifiable structure is acting for optic body power change with eye accommodation and dis-accommodation.

FIG. 10 shows front view of self-adjustable opto-fluidic switchable AIOL in free standing state with its optic body containing switchable optical element (SOE) as the power changing structure. The supporting structure composed of two units. One of these units is zeroing supporting structure to serve for AIOL size self-adjustment to match to a ciliary body ring diameter CBRD_{S} at dis-accommodation. Another unit is actuating supporting structure to serve for SOE power switching in response to ciliary body ring diameter changes with eye accommodation and dis-accommodation.

FIG. 11 demonstrates a cross-section of the switchable AIOL of the FIG. 10. It demonstrates a cross-section of the opto-fluidic switchable optical element to demonstrate substrate displacement with an interaction between the substrate extension and actuating modifiable structure.

FIG. 12 shows a block diagram of Installation mode of sizing self-adjustable AIOL of the present invention followed by Operation mode in which a power change of the optic body is coordinated with eye accommodation and dis-accommodation by ciliary muscle contraction and relaxation.

### DETAILED DESCRIPTION OF THE PREFERRED EMBODIMENTS

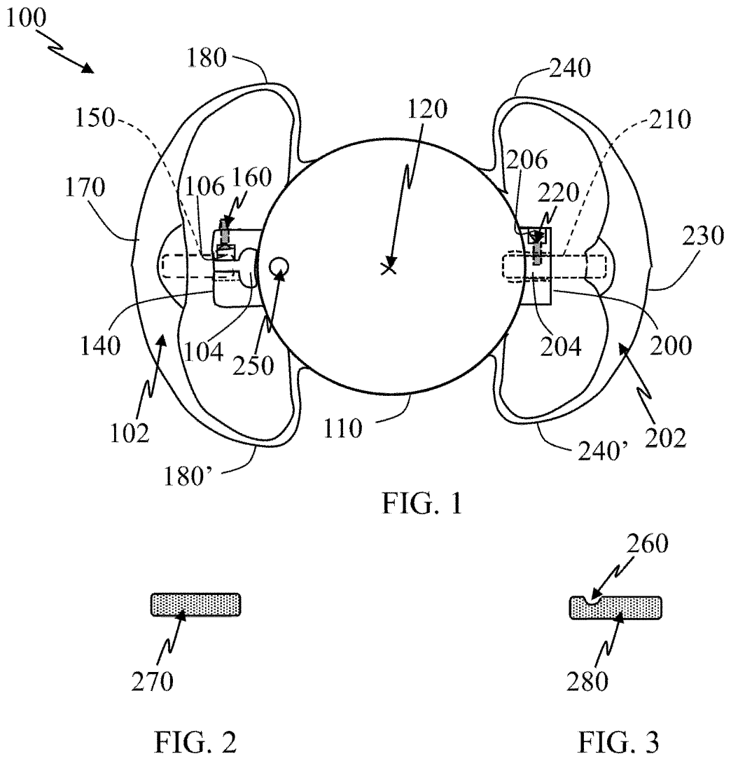

FIG. 1 shows a front view of an incomplete AIOL 100 in free standing state. It includes optic body 110 with optical axis 120 that includes power changing structure and two unit supporting structures. One is zeroing supporting structure 102 includes zeroing supporting element 170 which is in contact with ciliary body upon AIOL implantation. It is connected to the optical body 110 by zeroing deformable element 180 and 180′ in a form of elastic fiber loop that deforms for matching the AIOL size to an individual CBRD_{S} at dis-accommodation. Deformable element involves a shape change of the supporting structure. The AIOL 100 includes also zeroing locking structure 140 connected with the optic body 110 which includes zeroing stopper 160 disposed within a cavity 106 of the zeroing locking structure. The FIG. 1 references to the placement 150 of zeroing modifiable element for connecting the optic body 110 via locking structure 140 and zeroing supporting structure at the zeroing supporting element 170. Two, is an actuating supporting structure 202 that includes actuating supporting element 230 which is in contact with ciliary body upon AIOL implantation. It is connected to the optical body 110 by actuating deformable element 240 and 240′ in the form of elastic fiber loop which deforms with CBR_{D} change with accommodation. Zeroing and actuating supporting structures look similar and in order to distinguish them visually a hole 250 (or other indicia) is placed at the periphery of optic body 110. The AIOL 100 includes actuating locking structure 200 connected with the optic body 110 which includes actuating stopper 220 disposed in a cavity 206 of the actuating locking structure. The FIG. 1 references to actuating modifiable element placement 210 connecting the optic body 110 via actuating locking structure 200 and actuating supporting structure at the actuating supporting element 230.

FIG. 2 demonstrate an example of zeroing modifiable structure in a form of zeroing pusher 270 to be placed at incomplete AIOL of FIG. 1. It is in a form a rectangular cross-section and is placed at the zeroing modifiable element placement 150 and extends into a cavity 104 of the zeroing locking structure 140.

FIG. 3 demonstrates an example of actuating modifiable structure in a form of actuating pusher 280 at the incomplete AIOL of FIG. 1. It is in a form of a rectangular cross-section and is placed at the actuating modifiable element placement 210 and extends into a cavity 204 of the actuating locking structure 200. The actuating pusher 280 includes cut-out 260. It is understood by those skilled in the art that other cross-sectional shapes could be used for the zeroing pusher and actuating pusher, such as circular, square, triangular or any other polygonal shape.

A fabrication of the complete AIOL include separate fabrication of (1) incomplete AIOL per FIG. 1, (2) separate stoppers 160 and 220 to be inserted into the corresponding locking structures 140 and 200 and (3) zeroing pusher 270 and actuating pusher 280 to be inserted into the corresponding locking structures 140 and 200 at one end and are glued to the corresponding supporting elements 170 and 230 at the other ends.

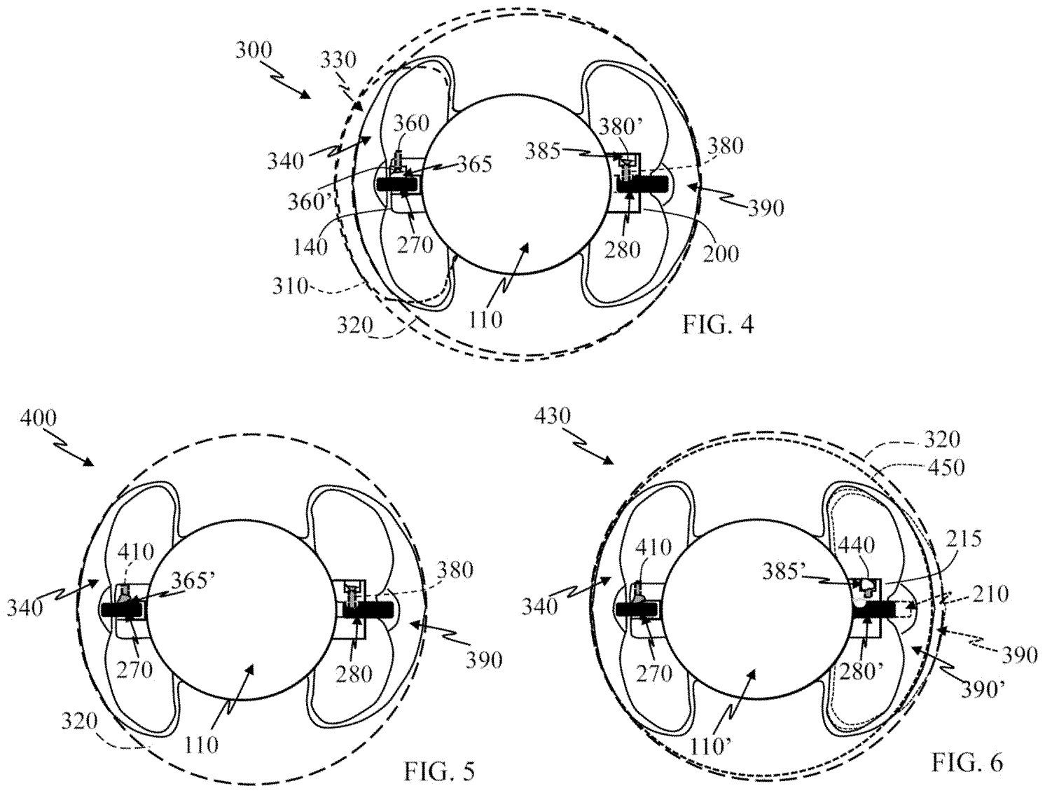

FIG. 4 shows sizing self-adjustable complete AIOL 300 of the FIG. 1 having size (free standing AIOL diameter) 310 circumvent zeroing supporting structure 330 and actuating supporting structure 390. The actuating pusher 280 is attached to the actuating supporting structure 390 at one end and its movement is prevented by the actuating stopper body 380 of the actuating locking structure 200 by protruding into the cut-out 260 of the actuating pusher 280. The AIOL is in first changeable state where actuating supporting structure 390 is immobilized by locking of the actuating modifiable structure in the form of actuating puncher 280. The zeroing pusher 270 is freely sliding within the zeroing locking structure 140 with a shape change of the attached to it zeroing supporting structure 340. The position of zeroing pusher 270 is controlled by zeroing supporting structure 340 which in turn depends upon CBRD_{S} 320 of the eye at dis-accommodation state. A change in position of the zeroing pusher 270 is allowed by the absent of contact with the zeroing stopper head 360′. The zeroing pusher does not interact with a power changing structure of optic body 110. Thus, the AIOL is in first changeable state. Zeroing locking structure 140 includes zeroing stopper consisting of zeroing stopper body 360 and zeroing stopper head 360′ located within the zeroing cavity 365. Actuating locking structure 200 includes actuating stopper consisting of zeroing stopper body 380 and actuating stopper head 380′ within the actuating cavity 385.

Shape change of the zeroing supporting structure might decenter the AIOL inside the eye from centration at free standing state but such decentration is expected to be small and should not exceed 1 mm. Besides, a width of the zeroing supporting structure in radial direction can be made slightly larger a width of the actuating supporting structure to compensate for zeroing supporting structure width reduction when placed in the eye.

FIG. 5 shows the AIOL 400 of the FIG. 4 in unchangeable state of the installation mode. The actuating supporting structure 390 remains immobilized by locking of the actuating modifiable structure in the form of actuating puncher 280 by the actuating stopper body 380. The zeroing supporting structure also becomes immobilized by locking the zeroing modifiable structure in the form of zeroing pusher 270 in place by zeroing stopper. By locking the zeroing pusher 270, the zeroing structure 340 is also immobilized. A process of locking might be based on a mechanically displacement of zeroing stopper to come into contact with zeroing pusher 270 but preferably it is performed remotely by a laser beam (first laser beam) melting the zeroing stopper head 360′ into shape 365′ that fills the zeroing cavity 365 thus coming into contact with the zeroing pusher 270 to lock it in place. This is called first modification of the modifiable structure from modifiable structure in first changeable state of AIOL 300 of FIG. 4 into unchangeable state of the AIOL 400 of FIG. 5. Thus, a size of the AIOL 400 matches the CBRD_{S} 320 at dis-accommodation of the individual patient. A power of the optic body 110 is maintained the same as in free standing AIOL 100 and AIOL 300 of FIG. 4 as the zeroing pusher 270 does not interact with the power changing structure of the optic body 110.

FIG. 6 shows the AIOL 430 where the AIOL 400 of FIG. 5 is undergone second modification into second changeable state to come into operation mode for patient to focus on objects at different distances with power change of optic body 110′. The zeroing supporting structure 340 remains immobilized by the locking of the zeroing modifiable structure in the form of zeroing pusher 270 by the zeroing stopper 410. The actuating supporting structure 390 becomes mobile by unlocking the actuating modifiable structure in the form of actuating pusher 280′ from blocking by the actuating stopper body. A process of unblocking might be based on a mechanical displacement of actuating stopper but preferably it is performed remotely by a laser beam (second laser beam) melting the actuating stopper head into shape 385′ that fills the actuating cavity which shorten the body of actuating stopper 440 as its material fills the actuating cavity. The resulted actuating stopper 440 does not interact with the actuating pusher 280′ thus allowing its free movement within the actuating locking structure 215. The actuating pusher is attached to the actuating supporting structure and a shape change between actuating supporting structure 390 to actuating supporting structure 390′ with the CBRD_{S} 320 in dis-accommodation change CBRD_{D} 450 at accommodation forces a sliding of the actuating pusher 280′ towards the optic body 110′ to engage with a power changing structure. This is called second modification of the modifiable structure as well as the corresponding AIOL from unchanged state of FIG. 5 to second changeable state of the FIG. 6.

Thus, the free moving actuating pusher 280′ interacts with a power structure of the optic body 110′ to change its power. Actuating pusher may press a fluid chamber of a fluidic power changing structure to control a power change, or it may move one of the wave plates controlling power change of power changing structure per Alvarez design, or it may trigger electric field change for power switching in electro-optical power changing structure and so on. The AIOL 430 ends up in operation mode to provide accommodation for a patient.

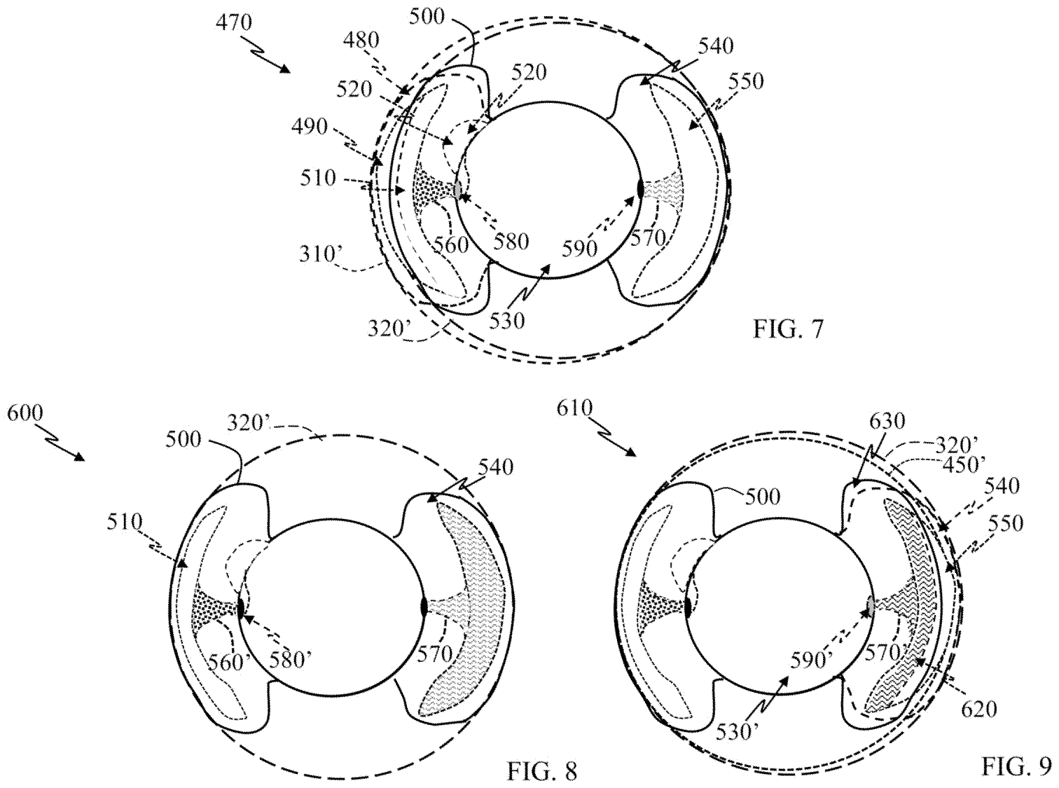

FIG. 7 shows sizing self-adjustable AIOL 470 in first changeable state for matching its size 310′ to CBR_{D} 320′. Its supporting structure is comprised of two units—zeroing supporting structure 480 of the AIOL in free standing state and actuating supporting structure 540. The zeroing supporting structure 480 includes zeroing deformable element in the form of a fluidic chamber 490 and the actuating supporting structure 540 includes actuating deformable element in the form of a fluidic chamber 550. Upon the AIOL placement in the eye in dis-accommodating state, the size of the AIOL forms to match the corresponding CBR_{D} 320′ as the zeroing supporting structure changes from the shape 480 to shape 500 with the correspond volume of zeroing deformable element 490 to the volume of zeroing deformable element 510 with a fluid flow through the zeroing modifiable structure 560 with unblocked zeroing stopper 580 to the out-flow chamber 520. The modifiable structure of the AIOL is made of zeroing modifiable structure 560 and actuating modifiable structure 570 connected with the actuating deformable element 550. Both modifiable structures may be in a form of a hollow tube for a fluid flow within it. The zeroing locking structure includes membrane 580 and out-flow chamber 520 with the zeroing modifiable structure 560 connected with out-flow chamber 520 via zeroing stopper 580 which may be a form of opened membrane. Zeroing modifiable structure 560 is not connected with a power changing structure of the optic body 530. The actuating modifiable structure 570 is connected with a power changing structure of the optic body 530 but the fluid flow within the actuating modifiable structure 570 is blocked by the actuating stopper 590 which can be in form of a closed membrane. The AIOL 470 is shown in first changeable state of the installation mode where a shape of the actuating supporting structure is fixed by blocking the actuating modifiable structure 570 and zeroing supporting structure 480 may adjust to a shape of zeroing supporting structure 500 for the AIOL size matching to CBRD_{S} 320′ at dis-accommodation.

FIG. 8 shows the AIOL 470 of the FIG. 7 undergone first modification to AIOL 600 to transfer the AIOL into unchangeable state of the installation mode —actuating modifiable structure 570 remains unchangeable and zeroing modifiable structure 500 is transferred into unchangeable state as well for AIOL size fixing to CBRD_{S} 320′ at dis-accommodation. First modification might be performance by modifying the zeroing stopper 580 into zeroing stopper 580′ by, for instance, melting by a laser beam the open membrane 580 into close membrane 580′ to block a fluid flow within the zeroing modifiable structure 560′. As a result, in addition to fixing a shape of the actuating supporting structure 540, a shape of the zeroing supporting structure 500 is also fixed with AIOL sizing matching the CBRD_{S} 320′ at dis-accommodation state.

FIG. 9 shows the AIOL of the FIG. 8 undergone second modification into AIOL 610 in second changeable state—the zeroing supporting structure 500 remains unchanged and the actuating modifiable structure 570′ is unblocked for a fluid flow from the actuating deformable element 550 by opening the actuating stopper 590′. It can be also performed be a laser beam to open a hole in the closed membrane of the actuating stopper 590′. In this second changeable state of the actuating modifiable structure 570′, a shape of the actuating supporting structure 540 in dis-accommodation state may change to a shape of the actuating supporting structure 630 at accommodation with a volume of the actuating deformable element 550 reducing to a volume of the actuating deformable element 620 with a reduction of CBRD_{S} 320′ at dis-accommodation to CBRD_{D} 450′ at eye accommodation. A fluid flow within the actuating modifiable structure 570′ now interacts with a power changing structure of optic body 530′ to increase its power for accommodating state. For instance, to steepen optical body shape with the addition of fluid if the power changing structure is composed of a fluidic chamber. Thus, the second modification of the modifiable structure brings it into operation mode where the AIOL offers the patient visual functioning to focus at different distances with eye accommodation.

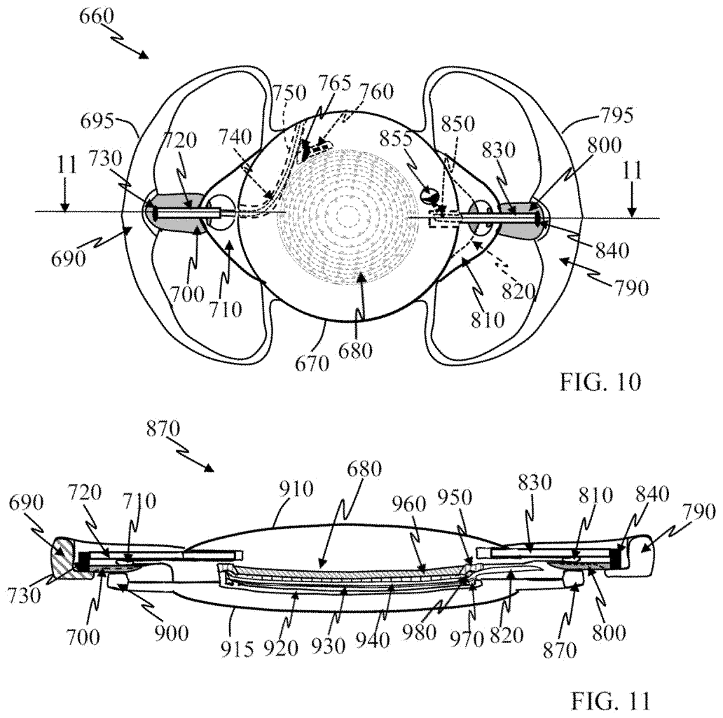

FIG. 10 shows self-adjustable opto-fluidic switchable AIOL 660 in free standing state prior to the installation in the eye. Optic body 670 contains power changing structure in a form of opto-fluidic switchable optical element (SOE) 680 described by Portney in the U.S. Patent Application No. 2021/0240010, which is incorporated in full herein with this reference. Like the AIOL of FIG. 1, the switchable AIOL 660 two-unit supporting structure includes unlocked for shape change zeroing structure 690 and locked actuating supporting structure 790. The zeroing supporting structure 690 is connected to a dual part zeroing modifiable element consisting of zeroing slider 700 and zeroing locker 720 which is extended by a flexible zeroing locker 740 inside the optic body 670. Both zeroing slider 700 and zeroing locker 720 are attached by element 730 to the zeroing support element 695 of the zeroing structure 690 at their peripheral ends. The other end of zeroing slider 700 is free to slide towards the optic body 670 guided between front and back zeroing guides which are part of zeroing locking structure. The FIG. 10 shows the front zeroing guide 710. Besides front and back zeroing guides, zeroing locking structure includes zeroing locking channel 750 within which the flexible zeroing locker 740 slides in AIOL first changeable state. The zeroing locking structure also includes zeroing stopper 760 and zeroing cavity 765 to serve for locking the zeroing stopper flexible part 750 in first modification to the AIOL unchangeable state. In this state the zeroing stopper position is locked thus also locking zeroing slider 700 position as well as a shape of zeroing supporting structure of the AIOL to match its size to the CBRD_{S} at dis-accommodation. Such first modification of the installation mode can be conducted similar to the process described in FIG. 5, i.e., by melting a head of zeroing stopper 760 to fill the zeroing cavity 765 to come in contact with the flexible zeroing locker 740 to lock it in position. The described configuration of the flexible zeroing locker 740 allows a placement of the locking stopper 760 closer to a center of optic body 670 for better accessibility to irradiation by the laser beam without extended patient's pupil dilation.

Like in FIG. 4, a shape of the actuating structure 790 is fixed by immobilizing the actuating modifiable structure. The actuating modifiable structure consists of actuating slider 800 and actuating locker 830 both attached to the actuating supporting element 795 by the element 840 at their peripheral ends. The other end of the actuating slider 800 is guided between the front and back actuating guides where the front actuating guide 810 is shown. The actuating locking structure includes front and back actuating guides, actuating locker 850 and actuating cavity 855. The AIOL of FIG. 10 is in first changeable state where the actuating locker 830 is locked in place by the actuating stopper 850 by their contact. The head of the actuating stopper within the actuating cavity 855 is targeted by laser irradiation for second modification of the actuating modifiable structure by unlocking the actuating locker 830 for free movement. The actuating stopper material is melted within the actuating cavity 855 to fill it by the actuating stopper material thus shortening the actuating stopper and terminating its contact with the actuating locker 830. The process brings the AIOL in second changeable state where the actuating supporting structure 790 is unlocked for a chape change with CBR_{D} change with accommodation and actuating slider 800 is unlocked for sliding towards the optic body 670. Actuating slider 800 sliding towards the optic body 670 engages the substrate extension 820 for the displacement of the SOE substrate-membrane combination inside the SOE 680 for power switching in coordination with eye accommodator. Thus, the AIOL goes through the process from installation mode (first changeable state and unchangeable state) to operation mode (second changeable state) where the patient experiences power switching for focusing at different distances.

FIG. 11 demonstrates a cross-section of the switchable AIOL 660 of FIG. 10. It shows the opto-fluidic switchable optical element SOE 680 imbedded between front optic body 910 and back optic body 915. The SOE consists of membrane cover 920, membrane 930 with non-matching fluid between them forming an external chamber, substrate 940 attached to the membrane 930 at the periphery to form active chamber in between filled with matching fluid, support ring 950 with plug 960 attached to it to form internal chamber with the substrate 940 filed with matching fluid and connected with the active chamber by multiple through holes at the substrate 940. The combination of substrate 940 and membrane 930 is attached to the membrane cover 920 at its periphery by membrane elastic element 970 and the combination is attached to the cover ring 950 by substrate elastic element 980 (both are ring shaped). Such elastic attachment of the substrate-membrane combination to the external parts of the SOE (support ring 950 and membrane cover 920) allows a displacement of the membrane-substrate combination within the switchable optical element 680 to transport the matching fluid out and in active change for SOE switching between refractive and diffractive states. The substrate 940 includes a substrate extension 820 to interact with the actuating modifiable structure, more specifically with actuating slider 800 when the switchable AIOL is in second changeable state of Operation mode.

In the second changeable state the zeroing supporting structure 690 has been already immobilized in unchangeable state by locked in place zeroing locker 720 which in turn immobilized the zeroing slide 700 within front part zeroing guide 710 and back part zeroing guide 900 as both zeroing slide 700 and zeroing locker 720 are attached to zeroing supporting structure by element 730. The actuating locker 830 is unlocked in second changeable state thus unlocking actuating supporting structure 790 and actuating slider 800. Thus, a reduction of the CBR_{D} from CBRD_{S} in dis-accommodation to CBRD_{D} in accommodation changes a shape of the actuating supporting structure 790 towards the optic body which in turn slides the actuating slide 800 guided by front part actuating guide 810 and back part actuating guide 870 towards the optic body. The actuating slider 800 engages the substrate extension 820 for down displacement thus pressing the combination of substrate 940 and membrane 930 towards the membrane cover 920. A displacement is allowed because of the combination of the substrate 940 and membrane 930 is suspended inside the SOE by the elastic elements 970 and 980. Such displacement increases pressure in the external chamber which in turn, presses the elastic membrane 930 against the diffractive surface of the substrate 940 and the matching fluid from the active chamber is transformed to internal chamber through holes in the substrate 940 as the total fluid volume inside the SOE is unchanged. As a result, the membrane surface shape is converted from continuous refractive shape into periodic diffractive shape defined by the diffractive surface of the substrate 940. Thus, the SOE converts from refractive state into diffractive state of different power. It is beneficial to design the diffractive power to be higher the refractive power and with accommodation the power of the switchable AIOL increases to focus near object on the retina.

With dis-accommodation and ciliary muscle relaxation, the size of the switchable AIOL increases due to elastic characteristic of the actuating structure 790 up for matching larger individual CBRD_{S}. It leads to a separation of the actuating slider 800 and substrate extension 820 to displace the combination of substrate 940 and membrane back towards the support ring 950. A pressure in the internal change increases transforming some matching fluid back into the active change between the substrate 940 and membrane 930 for refractive state.

A switching between power by displacement of a membrane-substrate combination may be converted from mechanical action of the position change by a modifiable structure as described above to electrical action with the use of smart material that changes its shape with electric field. It may be imbedded in one of the elastic elements 970 or 980 or acts separately on the substrate extension 820. For instance, by utilizing ionic polymer metal composite (IPMC). A typical IPMC consists of a polyelectrolyte membrane plated on both faces by a noble metal and is neutralized with certain counter ions that balance the electrical charge of the anions covalently fixed to the back-bone membrane.

The described above opto-fluidic switchable poticas element (SOE) design that includes elastic peripheral elements at both sides of the optical-membrane composition can be also applied to other applications such as a spectacle lens, contact lens and so on. The advantage is to allow for a displacement of the substrate-membrane combination inside the SOE to transport matching fluid in and out of the active change (a fluidic chamber between the substrate and membrane) for SOE switching between refractive and diffractive states of different powers. Such SEO may also include substrate extension for displacement actuation by an external mean or a displacement by imbedded in elastic peripheral support an actuator, IPMC for instance. IPMCs are active actuators that show large deformation in the presence of low applied voltage thus can be applied to switchable AIOL, contact lens, spectacle lens and other applications for power switching with opto-fluidic switchable optical element

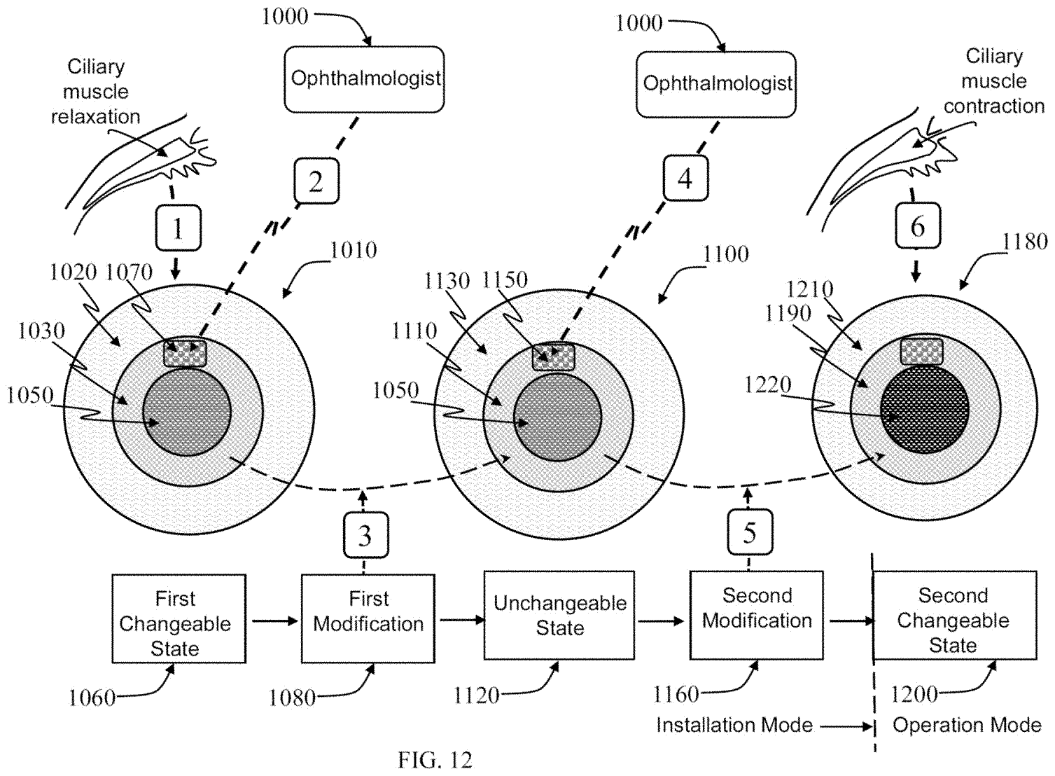

FIG. 12 illustrates a block diagram of installation and operation modes of a sizing self-adjustable AIOL of the present invention. U.S. Pat. No. 9,931,203 by Portney, the contents of which are fully incorporated herein with this reference, describes accommodating system that involves a sensor to sense a movement of ciliary muscle to produce a signal for system accommodation. The AIOL of the present invention does not involves a sensor at the ciliary muscle and all interaction occurs within the AIOL itself. The AIOL is placed inside the eye in first changeable state of Installation mode shown by block 1060. The eye is placed in dis-accommodation state where the ciliary muscle relaxes to form the largest ciliary body ring diameter (individual CBRD_{S}) of the patient. It results in the largest diameter AIOL that matches individual CBRD_{S} which is shown by line 1. The AIOL 1010 includes optic body 1050 with power changing structure for a power adjustment, modifiable structure 1030 in first changeable state where a change is in a form of position, shape, fluid flow or electric signal occurs with a change of the supporting structure 1020 and without interacting the power changing structure at the optic body 1050 for a power change.

A sizing self-adjustable AIOL of the present invention may be divided into two classes—mechanical AIOL where an interaction between supporting and modifiable structures occurs by a mechanical means such as a change in position, shape or fluid flow. The mechanical AIOL have been described by FIG. 1 through 11. Another class is electronic AIOL where an interaction between supporting and modifiable structures occurs by an electrical signal. In this case a change in shape of supporting structure effecting a modifiable structure of a mechanical AIOL is replaced by a change in electric signal of supporting structure effecting a modifiable structure of an electric AIOL. For instance, an electric signal is generated by a pressure sensor at a supporting structure. A modifiable structure of an electronic AIOL includes an amplifier, microprocessor, and memory. A change between the changeable and unchangeable states of AIOL is controlled by an ophthalmologist via wireless radio signal. A locking structure includes an RF receiver and convertor to transform a received RF signal into electric signal to program the modifiable structure for the changes between the changeable and unchangeable states.

Switching between changeable and unchangeable states has been described by FIG. 1 through 11 in case of a mechanical AIOL In case of an electronic AIOL, the AIOL is installed in the eye in first changeable state, block 1060, where eye is in dis-accommodation state with ciliary muscle relaxes, line 1. A pressure on a pressure sensor of the supporting structure 1020 changes with the individual CBRD_{S} till reaching a plateau level with matching AIOL size to the CBRD_{S} at dis-accommodation. During this process the modifiable structure 1030 does not communicate with the power changing structure of optic body 1050. An ophthalmologist 1000 send RF signal to locking structure 1070, line 2, which programs the modifiable structure 1030 that reached plateau signal constitutes the unchangeable state of the AIOL, block 1120, where there is no interaction with power changing structure and no further adjustment for the individual AIOL sizing to match CBRD_{S} at dis-accommodation. The process of programming the modifiable structure to record the corresponding plateau electric signal as the signal of the unchangeable state at the modifiable structure is shown by block 1080 and line 3.

To continue with electronic class of AIOL, the ophthalmologist 1000 sends RF signal, line 4, to communicate with locking structure 1150 for second modification, block 1160, of the modifiable structure 1110 as shown by line 5. It is to program the modifiable structure 1190 that an electric signal received from the supporting structure 1210 above the plateau signal recorded in unchangeable state is to communicate with the power changing structure of the optic body 1220 to change the power for intermediate or near focus with a reduction of CBR_{D} with ciliary muscle contraction, line 6. The electronic AIOL 1180 is in second changeable state, block 1200, and the Operation mode.

A mechanical AIOL first modification from first changeable state to unchangeable state followed by second modification of the Installation mode to bring a mechanical AIOL into second changeable of the operation mode as described by FIG. 1 through 11. The process follows the same block diagram of the FIG. 12.

What is claimed is:

1. An accommodative intraocular lens, comprising:
an optic body having an optical power changing structure configured for changing an optical power of the optic body;
two supporting structures disposed opposite one another about the optic body, each supporting structure of the two supporting structures configured to be connected at a distal end to a ciliary body after implantation in an eye of a patient and connected at a proximal end to the optic body, the two supporting structures being a zeroing supporting structure and an actuating supporting structure;
wherein the zeroing supporting structure is configured to not change the optical power of the optic body in an installation mode and an operation mode of the accommodative intraocular lens; and
wherein the actuating supporting structure is configured to not change the optical power of the optic body in the installation mode but is configured to change the optical power of the optic body in the operation mode of the accommodative intraocular lens.

2. The accommodative intraocular lens of claim 1, further comprising:
a zeroing locking structure attached to the optic body near the proximal end of the zeroing supporting structure;
a zeroing pusher attached to the distal end of the zeroing supporting structure and extending into a first cavity of the zeroing locking structure;
a zeroing stopper disposed within a second cavity of the zeroing locking structure, the second cavity being connected to the first cavity of the zeroing locking structure, wherein the zeroing stopper is not in contact with the zeroing pusher in a free standing state;
an actuating locking structure attached to the optic body near the proximal end of the actuating supporting structure;
an actuating pusher attached to the distal end of the actuating supporting structure and extending into a first cavity of the actuating locking structure;
an actuating stopper disposed within a second cavity of the actuating locking structure, the second cavity being connected to the first cavity of the actuating locking structure, wherein the actuating stopper is in contact with the actuating pusher preventing the actuating pusher from movement within the first cavity in the free standing state.

3. The accommodative intraocular lens of claim 2, wherein a first changeable state is when the zeroing stopper is in contact with the zeroing pusher preventing the zeroing pusher from movement within the first cavity of the zeroing locking structure.

4. The accommodative intraocular lens of claim 3, wherein the zeroing pusher is configured to make contact with the zeroing pusher by melting from a first laser beam of energy.

5. The accommodative intraocular lens of claim 3, wherein the zeroing stopper is configured to make contact with the zeroing pusher by a first mechanical displacement of the zeroing stopper.

6. The accommodative intraocular lens of claim 3, wherein a second changeable state is when the actuating stopper is no longer in contact with the actuating pusher allowing the actuating pusher movement within the first cavity of the actuating locking structure.

7. The accommodative intraocular lens of claim 6, wherein the actuating stopper is configured to disengage contact with the actuating pusher by melting from a second laser beam of energy.

8. The accommodative intraocular lens of claim 6, wherein the actuating stopper is configured to not make contact with the actuating pusher by a second mechanical displacement of the actuating stopper.

9. The accommodative intraocular lens of claim 2, wherein the zeroing supporting structure and the actuating supporting structure each comprise a deformable element being an elastic fiber loop.

10. The accommodative intraocular lens of claim 2, wherein the actuating pusher includes a cut-out configured for engagement by the actuating stopper in the free standing state.

11. The accommodative intraocular lens of claim 1, including a hole and/or indicia disposed on the optic body identifying the zeroing supporting structure as different from the actuating supporting structure.

12. The accommodative intraocular lens of claim 1, wherein the actuating pusher is configured to interact with the optical power changing structure comprising a fluidic chamber lens structure, a wave plate lens structure or an electro-optic lens structure.

13. The accommodative intraocular lens of claim 1, wherein the zeroing supporting structure comprises a zeroing fluidic chamber near its respective distal end and an out-flow chamber, and including a zeroing stopper allowing fluidic communication between the zeroing fluidic chamber and the out-flow chamber in the installation mode but blocking fluidic communication between the zeroing fluidic chamber and the out-flow chamber in the operation mode.

14. The accommodative intraocular lens of claim 13, wherein the actuating supporting structure comprises an actuating fluidic chamber near its respective distal end, and including an actuating stopper blocking fluidic communication between the actuating fluidic chamber and the optical power changing structure of the lens in the installation mode but allowing fluidic communication between the actuating fluidic chamber and the optical power changing structure of the lens in the operation mode.

15. The accommodative intraocular lens of claim 2, wherein the first cavity of the zeroing locking structure comprises a zeroing locking channel that extends at least partially non-linear through the optic body, wherein the zeroing pusher includes a flexible zeroing pusher portion that is disposed within the zeroing locking channel, and wherein the second cavity of the zeroing locking structure is connected to the zeroing locking channel.

16. An accommodative intraocular lens, comprising:
an optic body with a power changing structure for changing an optical power of the optic body;
a supporting structure configured to support the optic body inside an eye of a patient, the supporting structure configured to be in contact by a distal end with the ciliary body of the eye after implantation in the eye of the patient and connected at a proximal end to the optic body, the supporting structure is one of a zeroing supporting structure and an actuating supporting structure;
wherein the zeroing supporting structure is configured to not change the optical power of the optic body in an installation mode and an operation mode of the accommodative intraocular lens; and
wherein the actuating supporting structure is configured to not change the optical power of the optic body in the installation mode but is configured to change the optical power of the optic body in the operation mode of the accommodative intraocular lens.

17. An accommodative intraocular lens, comprising:
an optic body with a power changing structure for changing an optical power of the optic body;
a supporting structure configured to support the optic body inside an eye of a patient, the supporting structure configured to be in contact by a distal end with the ciliary body of the eye after implantation in the eye of the patient and connected at a proximal end to the optic body; and
a modifiable structure connected to the optic body and the supporting structure, wherein the modifiable structure:
is configured to not change the optical power of the optic body in an installation mode and an operation mode of the accommodative intraocular lens; and
is configured to not change the optical power of the optic body in the installation mode but is configured to change the optical power of the optic body in the operation mode of the accommodative intraocular lens.

18. A method of implanting the accommodative intraocular lens of claim 1 into the eye of the patient, the method comprising the steps of:
attaching the distal end of the zeroing supporting structure to a first portion of the ciliary body;
attaching the distal end of the actuating supporting structure to a second portion of the ciliary body;
locking the zeroing pusher in the first cavity of the zeroing locking structure by engaging the zeroing stopper with the zeroing pusher; and
unlocking the actuating pusher in the first cavity of the actuating locking structure by disengaging the actuating stopper with the actuating pusher.

19. A method of implanting the accommodative intraocular lens of claim 13 into the eye of the patient, the method comprising the steps of:
attaching the distal end of the zeroing supporting structure to a first portion of the ciliary body;
attaching the distal end of the actuating supporting structure to a second portion of the ciliary body;
blocking fluidic communication between the zeroing fluidic chamber and the out-flow chamber by changing a state of the zeroing stopper; and
allowing fluidic communication between the actuating fluidic chamber and the optical power changing structure by changing a state of the actuating stopper.
